(19) United States
(12) Patent Application Publication
(10) Pub. No.: US 2016/0136072 A1
(43) Pub. Date: May 19, 2016

(54) Use of special N-methyl-N-acylglucamines in skin-cleaning agents and hand dishwashing agents

(71) Applicant: CLARIANT INTERNATIONAL LTD, Muttenz (CH)

(72) Inventors: Peter KLUG, Grossostheim (DE); Carina MILDNER, Frankfurt am Main (DE); Henrike NEUHOFF, Wiesbaden (DE)

(73) Assignee: Clariant International Ltd., Muttenz (CH)

(21) Appl. No.: 14/901,090

(22) PCT Filed: Jun. 25, 2014
(86) PCT No.: PCT/EP2014/001722

§ 371 Date: Dec. 28, 2015

(30) Foreign Application Priority Data
Jun. 28, 2013 (DE) 10 2013 212 738.3
Nov. 29, 2013 (DE) 10 2013 224 559.9

(51) Int. Cl. A61K 8/42; A61K 8/41; C11D 1/14; C11D 3/32; C11D 1/90; A61K 8/46; A61Q 19/10

(52) U.S. Cl. CPC A61K 8/42; A61K 8/463; A61K 8/416; A61Q 19/10; C11D 3/32; C11D 1/90; C11D 1/146

(57) ABSTRACT

The invention relates to N-alkyl-N-acylglucamines of formula (I) suitable as a component in skin-cleaning agents and hand dishwashing agents, which comprise an aqueous surfactant system with at least one anionic surfactant, wherein, in formula (I), Ra is a linear or branched, saturated or unsaturated C₅-C₂₁ alkyl radical, preferably a C₁₁-C₁₇ alkyl radical, and Rb represents a C₁-C₄ alkyl radical, preferably methyl; the N-Alkyl-N-acylglucamines (I) containing at least 8 wt. %, based on the total amount of N-alkyl-N-acylglucamines (I), of compounds with a mono- or polyunsaturated C₁₈ fatty acid radical Ra—CO—.

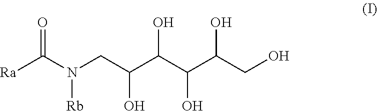

The invention relates to the use of specific N-alkyl-N-acylglucamines in skin cleansing compositions and also to skin cleansing compositions comprising these N-alkyl-N-acylglucamines.

Cosmetic cleansing products for the skin are subject to exacting requirements. They are to exhibit a good appearance, be toxicologically and ecotoxicologically unobjectionable, to provide excellent cleansing, while nevertheless generating a pleasant sensation on the skin. Hand dishwashing compositions are also subject to similar requirements.

The surfactant systems included in such products are required to fulfill a dual function. On the one hand, there is to be an adequate cleansing effect, which often, however, turns into a dull sensation on skin if degreasing is too strong. On the other hand, following use, the skin is to have a cared-for sensation.

These effects are normally achieved by adding additional skincare agents to the surfactant system present for cleansing.

These additional agents may be, for example, cationic surfactants (hydroxyethyl quats) or cationic polymers (polyquaternium-7, polyquaternium-10) or cationic guar derivatives, chitosan derivatives). Furthermore, fatty acid esters (glyceryl oleate) or ethoxylated fatty acid esters (PEG-7 glyceryl cocoate) are used for this purpose. All of these substances, however, have disadvantages in their use; the cationic components in particular are of only limited compatibility, owing to their cationic nature, with the commonly employed surfactant systems or other additives, such as opacifiers, for example.

EP-A 1 043 017 (DE 199 16 090) discloses skincare compositions such as creams, beauty lotions and sun protection products, which comprise N-acyl-N-alkylglucamides. The N-acyl-N-alkylglucamides are intended to take over the function of natural ceramides and make a contribution to the re-establishment of a disrupted skin barrier function. Influencing the skin sensation by means of such substances, however, is not described.

It has been found that certain N-methyl-N-acylglucamines, of the kind described in part in EP-A 1 043 017, when used in surfactant systems for skin cleansing, not only have good cleansing qualities but also contribute to improving the skin sensation after cleansing. It was surprising that in this way a surfactant is able to take over skincare functions in the beauty product, a purpose for which it is commonly necessary to add further additives.

A subject of the invention is therefore the use of N-alkyl-N-acylglucamines of the formula (I) in skin cleansing compositions and/or hand dishwashing compositions, in particular as a skincare component, which have an aqueous surfactant system comprising at least one anionic surfactant,

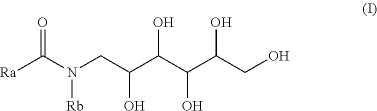

where in the formula (I)
Ra is a linear or branched, saturated or unsaturated C₅-C₂₁ alkyl radical, preferably C₁₁-C₁₇ alkyl radical, and
Rb is a C₁-C₄ alkyl radical, preferably methyl, and
the N-alkyl-N-acylglucamines (I) comprising at least 8 wt %, based on the total amount of N-alkyl-N-acylglucamines (I), of compounds having a singly or multiply unsaturated C₁₈ fatty acid radical Ra—CO—.

A further subject of the invention is a skin cleansing composition or hand dishwashing composition comprising
(a) one or more N-methyl-N-acylglucamines (I) containing at least 8 wt %, based on the total amount of N-alkyl-N-acylglucamines (I), of compounds having a saturated C₁₆, C₁₈, or singly or multiply unsaturated C₁₈ fatty acid radical Ra—CO—, as component (A),
(b) one or more anionic surfactants from the group of the alkyl ether sulfates, alkyl sulfates, and N-acylamino acid surfactants as component (B),
(c) optionally one or more betaine surfactants as component (C),
(d) optionally one or more further surfactants as component (D),
(e) one or more refatting agents as component (E),
(f) water as component (F), and
(g) optionally further additives, such as preservatives, fragrances, and dyes, as component (G).

Also subject of the invention is a method for cleansing the skin by contacting the skin with a skin cleansing composition comprising the inventive N-alkyl-N-acylglucamines (I).

The surfactant systems used in accordance with the invention, comprising long-chain glucamides, especially unsaturated glucamides, produce not only the cleansing effect but also a skincare effect, which is able to reduce or render superfluous the use of additional additives, such as further fatty acid esters, ethoxylated fatty acid esters, or cationic substances, and so contributes to a simplification of the overall formula.

The N-alkyl-N-acylglucamines (I) used in accordance with the invention, also known as N-alkyl-N-1-deoxysorbityl-fatty acid amides, contain at least 8, preferably at least 10, more preferably at least 15, very preferably at least 30, and especially preferably at least 60 wt % of N-alkyl-N-acylglucamines (I) having a singly or multiply unsaturated C₁₈ acyl radical CO—Ra.

Preferred N-alkyl-N-acylglucamines (I) are those for which the radical CO—Ra derives from lauric acid, palmitic acid, stearic acid, oleic acid, linoleic acid, or linolenic acid. Especially preferred are N-methyl-N-acylglucamines of the formula (I) where the acyl radical CO—Ra derives from lauric acid, palmitic acid, oleic acid, linoleic acid, or linolenic acid.

Preference extends to N-alkyl-N-acylglucamines (I) which contain at least 8 wt % of N-alkyl-N-acylglucamines (I) having a singly unsaturated C₁₈ fatty acid radical.

Where the N-alkyl-N-acylglucamines (I) used in accordance with the invention contain both unsaturated long-chain and medium-chain fatty acid radicals (C12), the resulting surfactant systems exhibit an outstanding combination of water solubility, cleansing performance, foam formation, and skincare properties. Fatty acid mixtures of this kind are possessed for example by natural palm kernel oil and coconut oil.

Also preferred, accordingly, are embodiments wherein the N-alkyl-N-acylglucamines (I) contain at least 8 wt % of N-alkyl-N-acylglucamines (I) having a singly or multiply unsaturated unsaturated C₁₈ fatty acid radical and at least 30 wt % having a saturated C₁₂ fatty acid radical.

The N-methyl-N-acylglucamines used in skin cleansing compositions in accordance with the invention preferably include only small fractions of N-methyl-N-acylglucamines containing C₆-C₁₀ acyl groups and derived from medium fatty acids. The fraction of such medium-length fatty acids is preferably not more than 15, more preferably 10, more particularly 5, wt %.

The N-methyl-N-acylglucamines (I) may be prepared, as described in EP-A 0 550 637 B1 and EP-A 0 285 768, by reaction of the corresponding fatty acid esters or fatty acid ester mixtures with N-methylglucamine in the presence of a solvent possessing hydroxyl groups or alkoxy groups. Examples of suitable solvents are C₁-C₄ monoalcohols, ethylene glycol, propylene glycol, glycerol, and alkoxylated alcohols. Preference is given to 1,2-propylene glycol. As likewise described in EP 0 550 637 A1, N-methylglucamine may be obtained by a reductive amination of glucose with methylamine. Suitable fatty acid esters reacted with the N-methylglucamines to give glucamides of the invention are generally the methyl esters, which are obtained by transesterification from natural fats and oils, the triglycerides for example.

Unsaturated C₁₈ acyl groups are understood in the sense of the invention to be fatty acid radicals having one or more double bonds. Preference in this context is given to radicals deriving from oleic acid, from linoleic acid, and from linolenic acid.

The aqueous surfactant systems used in accordance with the invention comprise one or more anionic surfactants, preferably from the group of the alkyl sulfates and alkyl ether sulfates, very preferably in combination with betaines.

In a further embodiment, the surfactant systems comprise fatty acid alkanolamides as well as alkyl ether sulfates and/or alkyl sulfates.

Preferred alkyl sulfates are the C₈-C₂₀ alkyl sulfates, more particularly the linear C₈-C₂₀ alkyl sulfates in the form of their sodium, potassium, or ammonium salts. Examples of alkyl sulfates are lauryl sulfate, cocoalkyl sulfate, and tallowalkyl sulfate. Lauryl sulfate is particularly preferred.

Preferred alkyl ether sulfates are the C₈-C₂₀ alkyl ether sulfates, more preferably the linear C₈-C₂₀ alkyl ether sulfates, more particularly the alkyl glycol ether sulfates derived from the ethoxylated fatty alcohols, in the form of their sodium, potassium, or ammonium salts. Examples of alkyl ether sulfates are lauryl ether sulfate, cocoalkyl ether sulfate, and tallowalkyl ether sulfate. Examples of glycol ether sulfates are lauryl triethylene glycol ether sulfate, cocoalkyl triethylene glycol ether sulfate, and tallowalkylhexaethylene glycol ether sulfate. Particular preference is given to lauryl glycol ether sulfate, as for example lauryldiethylene glycol ether sulfate or lauryltriethylene glycol ether sulfate, especially in the form of the sodium salts.

In a further preferred embodiment of the invention, the surfactant systems comprise one or more N-acylamino acid surfactants as anionic surfactants. In one preferred embodiment, the amino acid radical of such N-acyl-amino acid surfactants is selected from the group consisting of proteinogenic amino acids, their N-alkylated derivatives, or mixtures thereof.

Particularly preferred as N-acyl-amino acid surfactants are acylglycinates, acylalaninates, acylaspartates, acylglutamates, acylsarcosinates, or mixtures thereof. Especially preferred are the N-acyl-amino acid surfactants selected from the group consisting of acylglycinate, acylaspartate, acylglutamate, acylsarcosinate, and mixtures thereof.

With particular preference the N-acylamino acid surfactants consist of at least one C₈-C₂₂-acylated amino acid, more particularly the N-alkylated derivatives thereof. Preferred are the corresponding lauroyl or cocoyl derivatives of the amino acids.

Especially preferred accordingly are sodium cocoylglycinate, potassium cocoylglycinate, sodium lauroylglycinate, potassium lauroylglycinate, sodium cocoylglutamate, sodium lauroylglutamate, sodium cococylaspartate, sodium lauroylaspartate, and sodium lauroylsarcosinate.

The aqueous surfactant solutions preferably comprise a betaine surfactant (C) as well as the at least one anionic surfactant.

Betaine surfactants include within the same molecule a cationic group, especially an ammonium group, and an anionic group, which may be a carboxylate group, sulfate group, or sulfonate group. Suitable betaines are alkylbetaines such as cocobetaine or fatty acid alkylamidopropylbetaines, as for example cocoacylamidopropyldimethylbetaine, C₁₂-C₁₈ dimethylaminohexanoates, or C₁₀-C₁₈ acylamidopropanedimethylbetaines.

In one preferred embodiment of the invention, the aqueous surfactant systems comprise one or more amidopropylbetaines of the formula (II),

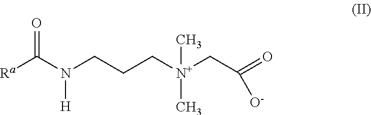

in which R^{a} is a linear or branched saturated C₇-C₂₁ alkyl group or a linear or branched singly or multiply unsaturated C₇-C₂₁ alkenyl group.

In a further preferred embodiment of the invention, the surfactant systems comprise one or more betaines of the formula (III),

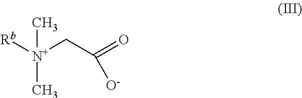

in which R^{b} is a linear or branched saturated C₈-C₂₂ alkyl group or a linear or branched singly or multiply polyunsaturated C₈-C₂₂ alkenyl group.

In a further preferred embodiment of the invention, the surfactant systems comprise one or more sulfobetaines of the formula (IV),

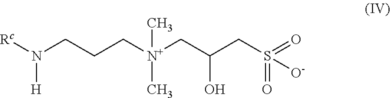

in which R^{c} is a linear or branched saturated C₈-C₂₂ alkyl group or a linear or branched singly or multiply unsaturated C₈-C₂₂ alkenyl group.

With particular preference, besides one or more alkyl sulfates and/or alkyl ether sulfates, the surfactant systems comprise one or more betaine surfactants selected from the group of the compounds consisting of the amidopropylbetaines of the formula (II), the betaines of the formula (III), and the sulfobetaines of the formula (IV).

In an especially preferred embodiment of the invention, the surfactant solutions comprise one or more betaine surfactants selected from the amidopropylbetaines of the formula (II).

In a further especially preferred embodiment of the invention, the surfactant solutions comprise one or more betaine surfactants selected from the betaines of the formula (III).

In a further especially preferred embodiment of the invention, the surfactant solutions comprise one or more betaine surfactants selected from the sulfobetaines of the formula (IV).

The radical R^{a} in the one or more amidopropylbetaines of the formula (II) is preferably a linear or branched saturated C₇-C₁₇ alkyl group. Among the linear and branched saturated alkyl groups R^{a}, the linear saturated alkyl groups are preferred.

With particular preference the amidopropylbetaines of the formula (II) are cocoamidopropylbetaines.

The radical R^{b} in the one or more betaines of the formula (II) is preferably a linear or branched saturated C₈-C₁₈ alkyl group and more preferably a linear or branched saturated C₁₂-C₁₈ alkyl group. Among the linear and branched saturated alkyl groups R^{b}, the linear saturated alkyl groups are preferred.

The radical R^{c} in the one or more sulfobetaines of the formula (IV) is preferably a linear or branched saturated C₈-C₁₈ alkyl group and more preferably a linear or branched saturated C₁₂-C₁₈ alkyl group. Among the linear and branched saturated alkyl groups R^{c}, the linear saturated alkyl groups are preferred.

The aqueous surfactant systems more preferably comprise amidopropylbetaines of the formula (I) and/or alkylbetaines of the formula (II).

Optional further surfactants (D) may be cationic, nonionic, or amphoteric surfactants.

Suitable cationic surfactants are substituted or unsubstituted, straight-chain or branched, quaternary ammonium salts of the type R¹N(CH₃)₃X, R¹R²N(CH₃)₂X, R¹R²R³N(CH₃)X or R¹R²R³R⁴NX. The radicals R¹, R², R³ and R⁴ may preferably, independently of one another, be unsubstituted alkyl having a chain length between 8 and 24 carbon atoms, more particularly between 10 and 18 carbon atoms, hydroxyalkyl having 1 to 4 carbon atoms, phenyl, C₂ to C₁₈ alkenyl, C₇ to C₂₄ aralalkyl, (C₂H₄O)ₓH, where x is from 1 to 3, alkyl radicals containing one or more ester groups, or cyclic quaternary ammonium salts. X is a suitable anion. Preferred are (C₈-C₂₂)-alkyltrimethylammonium chloride or bromide, particularly preferred are cetyltrimethylammonium chloride or bromide, di-(C₈-C₂₂)-alkyldimethylammonium chloride or bromide, (C₈-C₂₂)-alkyldimethylbenzylammonium chloride or bromide, (C₈-C₂₂)-alkyldimethylhydroxyethylammonium chloride, phosphate, sulfate, lactate, particularly preferred are distearyldimethylammonium chloride, di(C₈-C₂₂)-alkylamidopropyltrimethylammonium chloride and methosulfate.

The amount of the cationic surfactants in the compositions of the invention may be up to 10 wt %, based on the overall weight of the completed compositions. Compositions of the invention preferably contain no cationic polymers.

The following compounds can be considered as nonionic surfactants for example:

polyethylene oxide, polypropylene oxide, and polybutylene oxide condensates of alkylphenols. These compounds comprise the condensation products of alkylphenols having a C₆ to C₂₀ alkyl group, which may be either linear or branched, with alkene oxides. These surfactants are referred to as alkylphenol alkoxylates, e.g., alkylphenol ethoxylates.

Condensation products of aliphatic alcohols with 1 to 25 mol of ethylene oxide. The alkyl or alkenyl chain of the aliphatic alcohols may be linear or branched, primary or secondary, and contains generally 8 to 22 carbon atoms. Particular preference is given to the condensation products of C₁₀ to C₂₀ alcohols with 2 to 18 mol of ethylene oxide per mole of alcohol. The alcohol ethoxylates may have a narrow (“narrow range ethoxylates”) or a broad homolog distribution of the ethylene oxide (“broad range ethoxylates”). Examples of commercially available nonionic surfactants of this type are Tergitol® 15-S-9 (condensation product of a linear secondary C₁₁-C₁₅ alcohol with 9 mol of ethylene oxide), Tergitol® 24-L-NMW (condensation product of a linear primary C₁₂-C₁₄ alcohol with 6 mol of ethylene oxide, having narrow molar weight distribution). This product class likewise includes the Genapol® brands from Clariant.

Condensation products of ethylene oxide with a hydrophobic basis, formed by condensation of propylene oxide with propylene glycol. The hydrophobic moiety of these compounds preferably has a molecular weight between 1500 and 1800. The addition of ethylene oxide onto this hydrophobic moiety leads to an improvement in the water solubility. The product is liquid up to a polyoxyethylene content of about 50% of the total weight of the condensation product, which corresponds to a condensation with up to about 40 mol of ethylene oxide. Commercially available examples of this product class are the Pluronic® brands from BASF and the Genapol® PF brands from Clariant.

Condensation products of ethylene oxide with a reaction product of propylene oxide and ethylenediamine. The hydrophobic unit of these compounds consists of the reaction product of ethylenediamine with excess propylene oxide and generally has a molecular weight of 2500 to 3000. Ethylene oxide is added onto this hydrophobic unit up to a content of 40 to 80 wt % of polyoxyethylene and a molecular weight of 5000 to 11 000. Commercially available examples of this compound class are the Tetronic® brands from BASF and the Genapol® PN brands from Clariant.

Further suitable nonionic surfactants are alkyl- and alkenyl-oligoglycosides and fatty acid polyglycol esters or fatty amine polyglycol esters each having 8 to 20 and preferably 12 to 18 carbon atoms in the fatty alkyl radical, alkyl-oligoglycosides, alkenyl-oligoglycosides, and fatty acid N-alkylglucamides.

The amount of the nonionic surfactants in the compositions of the invention may be up to 10 wt %, based on the overall weight of the completed compositions.

In addition, the compositions of the invention may comprise amphoteric surfactants. These may be described as derivatives of long-chain secondary or tertiary amines which possess an alkyl group having 8 to 18 carbon atoms and in which a further group is substituted by an anionic group which imparts water solubility, as for example by a carboxyl, sulfate, or sulfonate group. Preferred amphoteric surfactants are N—(C₁₂-C₁₈)alkyl β-aminopropionates and N—(C₁₂-C₁₈)alkyl β-iminodipropionates in the form of alkali metal and mono-, di-, and trialkylammonium salts. Suitable further surfactants are also amine oxides. These are oxides of tertiary amines having a long-chain group of 8 to 18 carbon atoms and two usually short-chain alkyl groups having 1 to 4 carbon atoms. Preference is given here, for example, to the C₁₀ to C₁₈ alkyldimethylamine oxides, fatty acid amidoalkyl-dimethylamine oxide.

The amount of the amphoteric surfactants in the compositions of the invention may be up to 10 wt %, based on the overall weight of the completed compositions.

Refatting agents (E) used may be preferably lanolin and lecithin, unethoxylated and polyethoxylated or acylated lanolin derivatives and lecithin derivatives, polyol fatty acid esters, mono-, di-, and triglycerides (glyceryl oleate, PEG-7 glyceryl oleate) and/or fatty acid alkanolamides (Cocamide MEA, Cocamide DEA, Cocamide MIPA), the latter serving simultaneously as foam stabilizers. They are used preferably in amounts of 0.01 to 10.0 wt %, more preferably of 0.1 to 5.0 wt %, and especially preferably of 0.5 to 3.0 wt %. The refatting agent (e) is structurally different from the rest of the components, particularly the N-methyl-N-acylglucamines (I).

Auxiliaries and adjuvants (g) are, for example, preservatives, fragrances, and dyes.

Suitable preservatives are the preservatives listed in the relevant annex of the European cosmetics legislation, for example phenoxyethanol, benzyl alcohol, parabens, benzoic acid, and sorbic acid; a particularly suitable example is 1,3-bis(hydroxymethyl)-5,5-dimethylimidazolidine-2,4-dione (Nipaguard® DMDMH).

The amount of the preservatives in the compositions of the invention is generally from 0.1% to 2.0 wt %, based on the total weight of the complete compositions.

Fragrances used may be individual odorant compounds, examples being the synthetic products of the ester, ether, aldehyde, ketone, alcohol and hydrocarbon types. Odorant compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethylmethylphenyl glycinate, allyl cyclohexyl-propionate, styrallyl propionate, and benzyl salicylate. The ethers include, for example, benzyl ethyl ethers, the aldehydes include, for example, the linear alkanals having 8 to 18 carbon atoms, citral, citronellal, citronellyloxyacetaldehyde, hydroxycitronellal, lilial, and bourgeonal, the ketones include, for example, the ionones, alpha-isomethylionone and methyl cedryl ketone, the alcohols include anethole, citronellol, eugenol, geraniol, linalool, phenylethyl alcohol, and terpineol, and the hydrocarbons include primarily the terpenes and balsams. Preference is given to using mixtures of different odorants which together produce a pleasing scent note.

Fragrances used may also be natural odorant mixtures, as obtainable from vegetable or animal sources, e.g., pine oil, citrus oil, jasmine oil, lily oil, rose oil, or ylang-ylang oil. Essential oils of relatively low volatility, which are usually used as aromatic components, are also suitable as perfume oils, e.g., sage oil, chamomile oil, clove oil, melissa oil, mint oil, cinnamon leaf oil, linden blossom oil, juniperberry oil, vetiver oil, olibanum oil, galbanum oil, and ladanum oil.

The amount of the fragrances in the compositions of the invention is generally from 0 to 2 wt %, based on the total weight of the completed compositions.

The dyes and color pigments present in the compositions of the invention, both organic and inorganic dyes, may be selected from the corresponding positive list in the Cosmetics Regulation, or the EC list of cosmetic colorants. Also used advantageously are pearlescent pigments, for example pearl essence (guanine/hypoxanthine mixed crystals from fish scales) and nacre (ground bivalve shells), monocrystalline pearlescent pigments, for example bismuth oxychloride (BiOCl), layer-substrate pigments, for example mica/metal oxide, silver-white pearlescent pigments composed of TiO₂, interference pigments (TiO₂, different layer thickness), color luster pigments (Fe₂O₃), and combination pigments (TiO₂/Fe₂O₃, TiO₂/Cr₂O₃, TiO₂/Prussian blue, TiO₂/carmine).

The amount of the dyes and pigments in the compositions of the invention is generally from 0.01 to 1.0 wt %, based on the total weight of the completed compositions.

Another subject of the invention is a skin clansing composition comprising
(a) one or more N-methyl-N-acylglucamines (I) containing at least 8 wt %, based on the total amount of N-alkyl-N-acylglucamines (I), of compounds having a saturated C₁₆, C₁₈, or singly or multiply unsaturated C₁₈ fatty acid radical Ra—CO—, as component (A),
(b) one or more anionic surfactants from the group of the alkyl ether sulfates, alkyl sulfates, and N-acylamino acid surfactants as component (B),
(c) optionally betaine surfactants as component (C),
(d) optionally further surfactants as component (D),
(e) one or more refatting agents as component (E),
(f) water as component (F), and
(g) optionally further additives, such as preservatives, fragrances, and dyes, as component (G),
   the skin cleansing composition preferably containing no cationic polymers.

Preferred components a), b), c), d), e), g) correspond to those stated above.

Generally speaking, the skin cleansing compositions comprise
(a) 0.1 to 10.0 wt %, preferably 1 to 5 wt %, of component (A),
(b) 0.1 to 15 wt %, preferably 1 to 10 wt %, of component (B),
(c) 0 to 10 wt %, preferably 1 to 8 wt %, of component (C),
(d) 0 to 10 wt %, preferably 1 to 6 wt %, of component (D),
(e) 0.01 to 10 wt %, preferably 1 to 5 wt %, of component (E),
(f) 45 to 99.8 wt %, preferably 75 to 95 wt %, of component (F),
(g) 0 to 10 wt %, preferably 0.1 to 5 wt %, of component (G).

The skin cleansing compositions of the invention preferably comprise the above-described alkyl sulfates and/or alkyl ether sulfates and betaine surfactants.

In one preferred embodiment of the invention, the skin cleansing compositions of the invention are in the form of skin cleansing products such as showering preparations, hand soaps, and facial cleansers.

The invention is illustrated in detail by the examples which follow.

### EXAMPLES

The glucamides described hereinbelow were prepared according to EP 0 550 637 from the corresponding fatty acid methyl esters and N-methylglucamine in the presence of 1,2-propylene glycol as solvent, and were obtained in solid form comprising active substance and 1,2-propylene glycol (all figures in wt %).

TABLE 1
Preparation examples for N-methyl-N-acylglucamines

| | | | | 1,2- | |
|---|---|---|---|---|---|
| Prepa- | | | Active | Propylene | Melting |
| ration | Methyl | | substance | glycol | point |
| example | ester | Triglyceride | (%) | (%) | (° C.) |
| 1 | C12/14 | — | 90 | 10 | 85 |
| | (C12: 70%, | | | | |
| | C14 30%) | | | | |
| 2 | | Coconut oil | 90 | 10 | 50 |
| | | (C8: 6%; | | | |
| | | C10: 6%; | | | |
| | | C12: 48% | | | |
| | | C14: 20% | | | |
| | | C16: 10%; | | | |
| | | C18: 2%, | | | |
| | | C18′ = 8%) | | | |
| 3 | C12/18 | — | 90 | 10 | 70 |
| | unsaturated | | | | |
| | (C12; 60%, | | | | |
| | C14: 26%, | | | | |
| | C16: 4% | | | | |
| | C18: 1% | | | | |
| | C18′ (oleic | | | | |
| | acid): 8% | | | | |
| | C18″ = 1% | | | | |
| 4 | C16/18 | — | 80 | 20 | 45 |
| | unsaturated | | | | |
| | C16: 32% | | | | |
| | C18: 8% | | | | |
| | C18′ = 52% | | | | |
| | C18″ = 8% | | | | |

Aqueous surfactant systems comprising sodium lauryl ether sulfate (SLES) [degree of ethoxylation 2 EO] (Genapol® LRO Liq., Clariant), cocoamidopropylbetaine (Genagen® CAB 818, Clariant), and sugar surfactants, in the mass ratios according to the table below, were produced and adapted to a uniform viscosity of 5000 mPas by addition of sodium chloride. The pH was adjusted to 5.5. The total surfactant content in each case was 15%.

The resulting surfactant systems were subjected to sensory evaluation in skin wash tests.

TABLE 2
Examples of inventive uses/skin cleansing compositions

| | | | Skin | Skin |
|---|---|---|---|---|
| | | | sensation, | sensation, |
| Example | Composition | Ratio | wet | dry |
| Comparative | SLES/betaines | 7:3 | ◯ | ◯ |
| example 1 | | | greasy | degreased |
| Comparative | SLES/betaines/ | 6:3:1 | − | − |
| example 2 | preparation example 1 | | dull | strongly |
| | | | | degreased |
| Inventive | SLES/betaines/ | 6:3:1 | + | + |
| example 1 | preparation example 2 | | pleasant | refatting |
| Inventive | SLES/betaines/ | 6:3:1 | + | + |
| example 2 | preparation example 4 | | pleasant | refatting |
| Comparative | SLES/betaines/ | 6:3:1 | + | ◯ |
| example 3 | coco-glucosides | | pleasant | degreased |

As is apparent from inventive examples 1 and 2 and comparative examples 1-3, the glucamides from example 1-3, in contrast to the base system (comparative example 1) and to a glucamide with C12/14 chain fraction (comparative example 2), impart positive sensory effects to the skin. Comparable sugar surfactants (comparative example 3) likewise do not have this effect.

### Formulation Example 1

Showering preparation

| | |
|---|---|
| Sodium laureth sulfate (2 EO) | 8% |
| Cocoamidopropylbetaine | 3% |
| N-Alkyl-N-acylglucamine as per preparation example 2 | 2% |
| Cocoamide MEA | 0.5% |
| Sodium chloride | 0.5% |
| Fragrance | 0.5% |
| Preservative | q.s. |
| Water | ad 100% |

### Formulation Example 2

Showering preparation

| | |
|---|---|
| Sodium laureth sulfate (2 EO) | 8% |
| Cocoamidopropylbetaine | 3% |
| N-Alkyl-N-acylglucamine as per preparation example 4 | 2% |
| Cocoamide MEA | 0.5% |
| Sodium chloride | 0.5% |
| Fragrance | 0.5% |
| Polyquaternium-7 | 0.2% |
| Preservative | q.s. |
| Water | ad 100% |

### Formulation Example 3

Hand dishwashing composition

| | |
|---|---|
| Sodium laureth sulfate (2 EO) | 8% |
| Cocoamidopropylbetaine | 3% |
| N-Alkyl-N-acylglucamine as per preparation example 4 | 2% |
| Fragrance | 0.2% |
| Preservative | q.s. |
| Water | ad 100% |

The stated percentages correspond to weight % and are based on the amount of active component.

### Use Example

An inventive showering preparation formulation B was assessed for fragrance stabilization. This was done by preparing the formulation, adding fragrance, storing it in closed glass bottles for two weeks at 40° C., and then having a panel of three trained assessors evaluate the remaining fragrance intensity in comparison to a comparative formulation A stored at 25° C.

| | Comparative | |
|---|---|---|
| | formulation A | Formulation B |
| | Composition | Composition |
| Formulation | (wt %) | (wt %) |
| Sodium lauryl ether | 9 | 7.3 |
| sulfate | | |
| Cocoamidopropylbetaine | 3 | 2.75 |
| Glucamide as per | 0 | 0.92 |
| preparation example 3 | | |
| PEG-40 hydr. Castor oil | 0.2 | 0 |
| PEG-200 glyceryl | 0.5 | 0 |
| palmate | | |
| PEG-7 glyceryl cocoate | 0.5 | 0 |
| Sodium benzoate | 0.2 | 0.2 |
| Sodium salicylate | 0.2 | 0.2 |
| Polyquaternium-7 | 0.2 | 0.2 |
| “Waterlilly” fragrance | 0.5 | 0.5 |
| Glycerol | 0.8 | 0.8 |
| Water | ad 100 | ad 100 |
| Evaluation of the odor | Significantly reduced | Fragrance intensity |
| after two weeks' storage | fragrance intensity, | largely unchanged |
| time at 40° C. | head note degraded | |

The result of the experiment shows that formulation B, containing no ethoxylates with terminal OH groups such as PEG-40 hydr. Castor oil, PEG-200 glyceryl palmate, or PEG-7 glyceryl cocoate, but instead having a glucamide, exhibits a much better fragrance stability on hot storage. It can therefore be concluded that the use of glucamides rather than of ethoxylates having terminal OH groups, as a skincare component, solubilizer, or thickener, leads at the same time to an improvement in the fragrance stability.

1. A method for cleansing skin, comprising the step of contacting the skin with a composition comprising at least one N-alkyl-N-acylglucamine of the formula (I) and an aqueous surfactant system comprising at least one anionic surfactant, 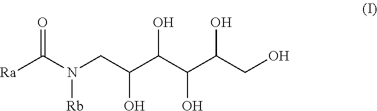
where in the formula (I)
Ra is a linear or branched, saturated or unsaturated C₅-C₂₁ alkyl radical and
Rb is a C₁-C₄ alkyl radical,
and wherein the at least one N-alkyl-N-acylglucamine according to formula (I) comprises at least 8 wt %, based on the total amount of the at least one N-alkyl-N-acylglucamine, of a singly or multiply unsaturated C₁₈ fatty acid radical Ra—CO—.

2. The method as claimed in claim 1, wherein Rb is a methyl radical.

3. The method as claimed in claim 1, wherein the radical R^{a} is derived from oleic acid, linoleic acid, or linolenic acid.

4. The method as claimed in claim 1, wherein the at least one N-alkyl-N-acylglucamine according to formula (I) comprises at least 15 wt % of a singly or multiply unsaturated C₁₈ fatty acid radical.

5. The method as claimed in claim 1, wherein the at least one N-alkyl-N-acylglucamine according to formula (I) comprises at least 8 wt % of a singly unsaturated C₁₈ fatty acid radical.

6. The method as claimed in claim 1, wherein the at least one N-alkyl-N-acylglucamine according to formula (I) comprises at least 8 wt % of an unsaturated C₁₈ fatty acid radical and at least 30 wt % of a saturated C₁₂ fatty acid radical.

7. The method as claimed in claim 1, wherein the aqueous surfactant system comprises an alkyl sulfate and/or an alkyl ether sulfate as anionic surfactant.

8. The method as claimed in claim 1, wherein the aqueous surfactant system comprises an alkyl sulfate and/or an alkyl ether sulfate as anionic surfactant and a betaine surfactant.

9. The method as claimed in claim 1, wherein the aqueous surfactant system comprises a linear C₈-C₂₀ alkyl sulfate and/or a linear C₈-C₂₀ alkyl ether sulfate.

10. The method as claimed in claim 9, wherein the aqueous surfactant system comprises lauryl sulfate and/or a lauryl ether sulfate.

11. The method as claimed in claim 1, wherein the aqueous surfactant system comprises an acylamidopropylbetaine or an alkylbetaine.

12. (canceled)

13. A skin cleansing composition or hand dishwashing composition comprising
(a) at least one N methyl-N-acylglucamine according to formula (I) containing at least 8 wt %, based on the total amount of N-alkyl-N-acylglucamine, of a saturated C₁₆, C₁₈, or singly or multiply unsaturated C₁₈ fatty acid radical Ra—CO—, as component (A), 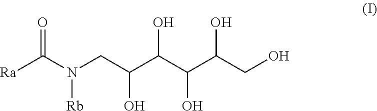
where in the formula (I)
Ra is a linear or branched, saturated or unsaturated C₅-C₂₁ alkyl radical and
Rb is a C₁-C₄ alkyl radical,
(b) at least one anionic surfactant selected from the group consisting of the alkyl ether sulfates, alkyl sulfates, and N-acylamino acid surfactants as component (B),
(c) optionally betaine surfactants as component (C),
(d) optionally further surfactants as component (D),
(e) one or more refatting agents as component (E),
(f) water as component (F), and
(g) optionally further additives, such as preservatives, fragrances, and dyes, as component (G).

14. The skin cleansing composition or hand dishwashing composition as claimed in claim 13, with the proviso that it contains no cationic polymers.

15. The skin cleansing composition or hand dishwashing composition as claimed in claim 13 comprising
(a) 0.1 to 10.0 wt % of component (A),
(b) 0.1 to 15 wt % of component (B),
(c) 0 to 10 wt % of component (C),
(d) 0 to 10 wt % of component (D),
(e) 0.01 to 10 wt % wt % of component (E),
(f) 45 to 99.8 wt % of component (F),
(g) 0 to 10 wt % of component (G).

16. The skin cleansing composition as claimed in claim 13, in the form of a showering preparation, a hand soap, or a facial cleanser.

17. A method for hand dishwashing, comprising the step of contacting the dish with a composition comprising at least one N-alkyl-N-acylglucamine of the formula (I) and an aqueous surfactant system comprising at least one anionic surfactant, 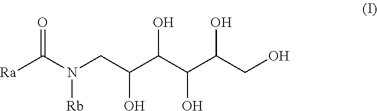
where in the formula (I)
Ra is a linear or branched, saturated or unsaturated C₅-C₂₁ alkyl radical and
Rb is a C₁-C₄ alkyl radical,
and wherein the at least one N-alkyl-N-acylglucamine according to formula (I) comprises at least 8 wt %, based on the total amount of the at least one N-alkyl-N-acylglucamine, of a singly or multiply unsaturated C₁₈ fatty acid radical Ra—CO—.

18. The method as claimed in claim 17, wherein Rb is a methyl radical.

19. The method as claimed in claim 17, wherein the radical R^{a} is derived from oleic acid, linoleic acid, or linolenic acid.

20. The method as claimed in claim 17, wherein the at least one N-alkyl-N-acylglucamine according to formula (I) comprises at least 15 wt % of a singly or multiply unsaturated C₁₈ fatty acid radical.

21. The method as claimed in claim 17, wherein the at least one N-alkyl-N-acylglucamine according to formula (I) comprises at least 8 wt % of a singly unsaturated C₁₈ fatty acid radical.

22. The method as claimed in claim 17, wherein the at least one N-alkyl-N-acylglucamine according to formula (I) comprises at least 8 wt % of an unsaturated C₁₈ fatty acid radical and at least 30 wt % of a saturated C₁₂ fatty acid radical.

23. The method as claimed in claim 17, wherein the aqueous surfactant system comprises an alkyl sulfate and/or an alkyl ether sulfate as anionic surfactant.

24. The method as claimed in claim 17, wherein the aqueous surfactant system comprises an alkyl sulfate and/or an alkyl ether sulfate as anionic surfactant and a betaine surfactant.

25. The method as claimed in claim 17, wherein the aqueous surfactant system comprises a linear C₈-C₂₀ alkyl sulfate and/or a linear C₈-C₂₀ alkyl ether sulfate.

26. The method as claimed in claim 25, wherein the aqueous surfactant system comprises lauryl sulfate and/or a lauryl ether sulfate.

27. The method as claimed in claim 17, wherein the aqueous surfactant system comprises an acylamidopropylbetaine or an alkylbetaine.
